(19) United States
(12) Patent Application Publication
(10) Pub. No.: US 2003/0228333 A1
(43) Pub. Date: Dec. 11, 2003

(54) Substituted hydrocarbyl functional siloxanes for household, health, and personal care applications

(76) Inventors: Cassandre Michelle Fecht, Sanford, MI (US); Deborah Lynn Meyers, Midland, MI (US); Heidi Marie Van Dort, Sanford, MI (US); Isabelle Van Reeth, Incourt (BE)

Correspondence Address: DOW CORNING CORPORATION CO1232, 2200 W. SALZBURG ROAD, P.O. BOX 994, MIDLAND, MI, 48686-0994, US

(21) Appl. No.: 10/157,639

(22) Filed: May 28, 2002

(51) Int. Cl. A61K 7/16; A61K 7/42; A61K 7/135; A61K 31/695; A61K 7/06

(52) U.S. Cl. 424/401; 424/59; 514/63; 424/62; 424/49

(57) ABSTRACT

Cosmetic, household, and medical compositions containing certain substituted hydrocarbyl functional siloxane fluids or certain substituted hydrocarbyl functional siloxane resins provide good aesthetic, solvency, and stability performance, and are useful as delivery vehicles for personal, health, and household care ingredients such as pigments, antiperspirant salts, drugs, sunscreens, alpha-hydroxy fatty acids, and vitamins.

### FIELD OF THE INVENTION

In the household, health and personal care areas, the need exists for silicone raw materials that contain both hydrophilic and hydrophobic functionality. To date, this need has been addressed with polyoxyalkylene and silanol functional silicone materials. These structures have a number of limitations including unpleasant aesthetics and acid/base instability, respectively.

The present invention relates to cosmetic, household and healthcare compositions containing substituted hydrocarbyl functional siloxane structures for improved performance and/or stability. In an alternate embodiment, certain substituted hydrocarbyl functional organosiloxane resins can be included in the compositions.

### BACKGROUND OF THE INVENTION

Polar silicone compounds have found use in a number of different types of products ranging from cosmetics to car polish formulations. Most of these products have been formulated as aerosol or pump sprays, liquids, roll-on liquids, creams, emulsions, gels, gel-solids, or solid stick formulations, and sometimes comprise an additional active material, e.g. vitamins,

alpha-hydroxy-acids, zirconium salts or combinations thereof, incorporated into a suitable carrier.

These products are designed to provide a variety of benefits, for example, moisturize dry skin, mask surface imperfections, and allow effective perspiration and odor control while being cosmetically acceptable during and after application onto the axillary region or other areas of the skin. Polar silicone compounds used in these applications include polydiorganosiloxane-polyoxyalkylene copolymers and silanol functional substituted siloxane structures.

The preparation of siloxane-oxyalkylene copolymers by hydrosilylation of an organohydrogensiloxane (SiH) and an olefinically substituted polyoxyalkylene is well known and reported in the literature. The hydrosilylation reaction is typically performed in a low molecular weight volatile hydrocarbon solvent such as benzene, toluene, xylene, or isopropanol to aid in handling the reactants, to moderate an exothermic reaction or to promote the solubility of the reactants. The CPA catalyzed reaction of Si—H polymers with allyl ethers was disclosed in U.S. Pat. No. 2,823,218 (Feb. 11, 1958). Typically, although not exclusively, the smallest oxyalkylene substitutents disclosed in patents are EO₄ and PO₂. This is because of the ready availability of the starting materials and synthetic ease of making allyloxypolyethers with that minimum substitution.

While U.S. Pat. No. 5,486,566 (Jan. 23, 1996) and U.S. Pat. No. 6,060,044 (May 9, 2000) describe silicone ethers containing only a single oxyalkylene unit in any chain, the compositions containing such silicone ethers must necessarily include guar gum or an hydroxystearic acid derivative for effectiveness, whereas compositions according to this invention possess the above described benefits without requiring the use of guar gum and hydroxystearic acid derivatives.

### SUMMARY OF THE INVENTION

This invention relates to a composition which is a guar gum free and hydroxystearic acid derivative free mixture of (i) a substituted hydrocarbyl functional siloxane fluid, or a substituted hydrocarbyl functional siloxane resin; and (ii) a cosmetic ingredient, a household care ingredient, or a health care ingredient, and (iii) an optional cosmetic active, a household care active, or a health care active such as an antiacne agent, anticaries agent, antidandruff agent, antifungal agent, antimicrobial agent, antioxidant, antiperspirant agent, cosmetic biocide, deodorant agent, external analgesic, oral care agent, oral care drug, oxidizing agent, reducing agent, skin bleaching agent, skin protectant, sunscreen agent, UV light absorbing agent, pigments, moisturizers, vitamins, enzymes, optical brighteners, fabric softening agents, or surfactants.

These and other features of the invention will become apparent from a consideration of the detailed description.

### DETAILED DESCRIPTION OF THE INVENTION

The present invention is based on the unexpected discovery that the inclusion of a substituted hydrocarbyl functional siloxane, in particular, results in personal care, medical and household care compositions with novel properties. For example, compositions containing such siloxanes impart the following characteristics when compared to similar formulations without such siloxanes: (a) remain stable at relatively high and low pH; (b) compatibilize both polar and non-polar solvents; (c) offer high lubricity; (d) mitigate skin discomfort or irritation; (e) impart a smooth, soft, moist texture; (f) evenly disperse active agents and pigments; (g) detackify formulation components; (h) moisturize the skin; (i) enhance the durability of formulation components; (j) enhance surface shine; (k) impart the sensory performance of higher molecular weight structures; (l) protect the hair cuticle; (m) aid curl retention; (n) sustain fragrance release; (o) impart softness to solid substrates; (p) increase water absorbency of fabrics; (q) mask surface imperfections; (r) reduce whitening of antiperspirant salts; (s) modify formulation rheology; (t) improve particulate active suspension; (u) improve ease of ironing; (v) enable the suspension and delivery of polar materials in non-polar solvents; and (w) enhance emulsion stability.

As used herein, the terms personal care composition, health care composition, and household care composition are intended to mean typical materials commercially available as products or raw materials in consumer markets containing active and inactive ingredients.

In the preferred embodiment, the compositions of the present invention comprise at least one substituted hydrocarbyl functional siloxane. These siloxanes generally have a formula selected from the group consisting of:

R₃SiO(RR¹SiO)_{y}SiR₃,

R₃ SiO(R₂SiO)ₓ(RR¹SiO)_{y}SiR₃,

R¹R₂SiO(R₂SiO)ₓSiR₂R¹,

R¹R₂SiO(RR¹SiO)_{z}SiR₂R¹,

R¹ R₂SiO(R₂SiO)ₓ(RR¹SiO)_{z}SiR₂R¹,

R¹R₂SiO(R₂SiO)ₓSiR₃,

R¹R₂SiO(RR¹SiO)_{z}SiR₃,

R¹R₂SiO(R₂SiO)ₓ(RR¹SiO)_{z}SiR₃, and cyclic siloxanes of the formula

—(Me₂SiO)ₘ(MeR¹SiO)ₙ—.

In these formulas, R is an alkyl, cycloalkyl, alkenyl, aralkyl, or an aryl group containing 1-20 carbon atoms; R¹ is a group having one of the formulas (i)-(iv):

—(CH₂)ₐ(OCH₂CH₂)OR² (i)

—(CH₂)ₐ(OCH₂CH₂CH₂)OR² (ii)

—(CH₂)ₐ[OCH₂CH(CH₂CH₃)]OR² (iii)

—(R)_{b}OR² (iv)

wherein a is 3-11; b is 1-50; R² is selected from the group consisting of hydrogen, an alkyl group, an aryl group, an arylalkyl group and an acyl group; x is 1-500, y is 1-40, z is 1-40, m is 1-6, n is 1-6, and the sum of m+n is 3-12.

For the sake of simplicity in nomenclature in the Examples, these substituted hydrocarbyl siloxanes are referred to as the CARBINOL FLUID. In preferred embodiments of the invention, and in the Examples, R is the methyl group, R¹ is —(CH₂)ₐ(OCH₂CH₂)OR²,

R² is hydrogen, a is 3, x is

3-500, y and z are 10-20, and m+n is 5.

In the alternate embodiment, compositions of the present invention can contain a substituted hydrocarbyl functional siloxane resin rather than a substituted hydrocarbyl functional siloxane fluid. Suitable substituted hydrocarbyl functional siloxane resins have the formula

(SiO₂)_{c}(R³ SiO_{3/2})_{d}(R³₂SiO)ₑ(R³₃SiO_{1/2})_{f}{O_{1/2}SiR³₂R⁴}_{g}

where R³ is an alkyl group with 1-20 carbon atoms, a cycloalkyl group with 3-20 carbon atoms, an alkenyl group with 2-20 carbon atoms, an aralkyl group, or an aryl group; R⁴ is the same as R¹ above, i.e., one of the formulas (i) to (iv); and g is 1-15,000. In such resins, c, d, e, and f represent mole percents, such that c<100, c+d>0, and c+d+e+f is 100. Organosiloxane resins of this type typically contain about 0.01-15 weight percent of silanol.

As used herein, the term hydrocarbyl is defined as any group consisting exclusively of carbon and hydrogen. The hydrocarbyl group can be branched or unbranched, saturated or unsaturated, and can contain one or more rings. Some suitable hydrocarbyl groups include alkyl, alkenyl, alkynyl, and aryl groups. Also included are alkyl, alkenyl, alkynyl, and aryl groups which are substituted with other aliphatic or cyclic hydrocarbyl groups such as alkaryl, alkenaryl, and alkynaryl.

The term substituted hydrocarbyl is therefore intended to mean any such hydrocarbyl group wherein at least one hydrogen atom has been substituted with an atom other than hydrogen, or with a group of atoms containing at least one atom other than hydrogen. For example, the hydrogen atom can be substituted with a halogen atom such as a chlorine or fluorine atom. The hydrogen atom alternatively can be substituted with an oxygen atom, or with a group containing an oxygen atom to form a hydroxy group, an ether, an ester, an anhydride, an aldehyde, a ketone, or a carboxylic acid. The hydrogen atom also can be replaced with a group containing a nitrogen atom to form an amide or a nitro group. In addition, the hydrogen atom can be substituted with a group containing a sulfur atom to form —SO₃H.

The substituted hydrocarbyl functional siloxanes of the present invention can be made by standard processes such as the hydrosilylation of organohydrogensiloxanes and olefinically substituted polyoxyalkylenes. The hydrosilylation reaction is typically performed in a low molecular weight volatile hydrocarbon solvent such as benzene, toluene, xylene, or isopropanol to aid in handling the reactants, to moderate an exothermic reaction or to promote the solubility of the reactants. Such processes are described, for example, in the '218 patent noted above, which is incorporated herein by reference.

These silicone compounds are useful in a number of different products, including hair care products such as hairsprays, shampoos, mousses, styling gels and lotions, cream rinses/conditioners, hair tonics, hair dyes and colorants, permanent waves and bleaches. Also included are skin care products such as cleansers, moisturizers, conditioners, lipsticks, eye makeup, foundations, fingernail polish, suntan products, antiperspirant/deodorant products and depilatories. Also included are household products such as waxes, polishes, heavy and light duty liquid cleaners, fabric softeners, ironing aids, laundry detergents, and window cleaners.

Some typical ingredients used in these products are surfactants, pigments, solvents, emollients, and carriers. For example, the solvents can include esters (for example, isopropyl myristate and C₁₂₋₁₅ alkyl lactate), water, silicone fluids (for example, cyclomethicone, dimethicone), ethanol, isopropanol, guerbet alcohols having 8-30 carbons, particularly 12-22 carbons (for example, isolauryl alcohol, isocetyl alcohol, isostearyl alcohol), fatty alcohols (for example, stearyl alcohol, myristyl alcohol, oleyl alcohol), and ethoxylated and propoxylated alcohols (for example, the polyethylene glycol ether of lauryl alcohol that conforms to the formula CH₃(CH₂)₁₀CH₂(OCH₂CH₂)ᵣOH where r has an average value of 4 (Laureth-4); PPG-14 butyl ether, where the “PPG-14” portion is the polymer of propylene oxide that conforms generally to the formula H(OCH₂C(CH₃)H)ₛOH, where s has an average value of 14, or PPG-3 myristyl ether which is the polypropylene glycol ether of myristyl alcohol that conforms to the formula CH₃(CH₂)₁₂CH₂(OCH(CH₃)CH₂)ₜOH where t has an average value of 3, or a hydrocarbon fluid.

Hydrocarbon fluids are exemplified by organic hydrocarbon fluids such as halogenated hydrocarbon fluids, aliphatic hydrocarbon fluids, aromatic hydrocarbon fluids, and mixtures of aromatic and aliphatic hydrocarbon fluids. The hydrocarbon fluids usually contain about 6 to about 12 carbon atoms. Examples of suitable hydrocarbon fluids include perchloroethylene, benzene, xylene, toluene, mineral oil fractions, kerosenes, naphthas, and petroleum fractions. Particularly preferred are isoparaffinic hydrocarbon fluids exemplified by isoparaffin fluids available from Exxon Mobil Chemical Company, Houston, Tex. U.S.A, sold as Isopar® M Fluid (a C₁₃-C₁₄ Isoparaffin), Isopar® C Fluid (a C₇-C₈ Isoparaffin), Isopar® E Fluid (a C₈-C₉ Isoparaffin), Isopar® G Fluid (a C₁₀₋₁₁ Isoparaffin), Isopar® L Fluid (a C₁₁-C₁₃ Isoparaffin), Isopar® H Fluid (a C₁₁-C₁₂ Isoparaffin), and combinations thereof. Mixtures of solvents can also be used.

Another ingredient which can be used is an emollient, including compositions such as guerbet alcohols (such as isocetyl alcohol or isostearyl alcohol); esters (such as isopropyl palmitate, isopropyl isostearate, octyl stearate, hexyl laurate and isostearyl lactate); a liquid mixture of hydrocarbons which are liquids at ambient temperatures (such as petroleum distillates and light mineral oils); ethanol; volatile and non-volatile silicone oils, highly branched hydrocarbons, and non-polar carboxylic acids. The emollients can be included in the compositions of the present invention in amounts within the range of 0.01-70%, preferably 0.1-25%, by weight, of the total weight of the composition.

The carrier can include a wide variety of conditioning materials, such as hydrocarbons, silicone fluids, and cationic materials. The carrier can include surfactants, suspending agents, thickeners etc. Various additional components useful in these compositions are described in U.S. Pat. No. 4,387,090 (Jun. 7, 1983).

Topical cosmetic, and pharmaceutical compositions according to the invention can contain a carrier, but the carrier should be cosmetically and/or pharmaceutically acceptable, i.e., that it is suitable for topical application to the skin, has good aesthetic properties, is compatible with the siloxane copolymers of the present invention, and will not cause any safety or toxicity concerns. It can be formulated to include an emulsion as the carrier such as an oil-in-water emulsion, water-in-oil emulsion, water-in-oil-in-water emulsion, or oil-in-water-in-silicone oil emulsion.

Some other suitable topical carriers include anhydrous liquid solvents such as oils, alcohols, and silicones (e.g., mineral oil, ethanol, isopropanol, dimethicone, cyclomethicone, and the like); aqueous-based single phase solvents (e.g., where the viscosity of the solvent has been increased to form a solid or semi-solid by the addition of appropriate gums, resins, waxes, polymers, salts, and the like). However, the preferred cosmetically and/or pharmaceutically acceptable topical carrier is a hydroalcoholic system or an oil-in-water emulsion. When the carrier is an oil-in-water emulsion, it will include common ingredients generally used for preparing emulsions.

Some of the typical active ingredients used in products such as these are antiacne agents, anticaries agents, antidandruff agents, antifungal agents, antimicrobial agents, antioxidants, antiperspirant agents and deodorant agents, cosmetic biocides, external analgesics, oral care agents, oral care drugs, oxidizing agents, reducing agents, skin bleaching agents, skin protectants, sunscreen agents, UV light absorbing agents, enzymes, optical brighteners, fabric softening agents, and surfactants

Some examples of antiacne agents are Salicylic acid and Sulfur. Some examples of anticaries agents are Sodium Fluoride, Sodium Monofluorophosphate, and Stannous Fluoride. Some examples of antidandruff agents are Coal tar, Salicylic acid, Selenium Sulfide, Sulfur, and Zinc Pyrithione. Some examples of antifungal agents are Calcium Undecylenate, Undecylenic Acid, Zinc Undecylenate, and Povidone-Iodine. Some examples of antimicrobial agents are Alcohol, Benzalkonium Chloride, Benzethonium Chloride, Hydrogen Peroxide, Methylbenzethonium Chloride, Phenol, Poloxamer 188, and Povidone-Iodine.

Some examples of antioxidants are Acetyl Cysteine, Arbutin, Ascorbic Acid, Ascorbic Acid Polypeptide, Ascorbyl Dipalmitate, Ascorbyl Methylsilanol Pectinate, Ascorbyl Palmitate, Ascorbyl Stearate, BHA, p-Hydroxyanisole, BHT, t-Butyl Hydroquinone, Caffeic Acid, Camellia Sinensis Oil, Chitosan Ascorbate, Chitosan Glycolate, Chitosan Salicylate, Chlorogenic Acids, Cysteine, Cysteine HCI, Decyl Mercaptomethylimidazole, Erythorbic Acid, Diamylhydroquinone, Di-t-Butylhydroquinone, Dicetyl Thiodipropionate, Dicyclopentadiene/t-Butylcresol Copolymer, Digalloyl Trioleate, Dilauryl Thiodipropionate, Dimyristyl Thiodipropionate, Dioleyl Tocopheryl Methylsilanol, Isoquercitrin, Diosmine, Disodium Ascorbyl Sulfate, Disodium Rutinyl Disulfate, Distearyl Thiodipropionate, Ditridecyl Thiodipropionate, Dodecyl Gallate, Ethyl Ferulate, Ferulic Acid, Hydroquinone, Hydroxylamine HCI, Hydroxylamine Sulfate, Isooctyl Thioglycolate, Kojic Acid, Madecassicoside, Magnesium Ascorbate, Magnesium Ascorbyl Phosphate, Melatonin, Methoxy-PEG-7 Rutinyl Succinate, Methylene Di-t-Butylcresol, Methylsilanol Ascorbate, Nordihydroguaiaretic Acid, Octyl Gallate, Phenylthioglycolic Acid, Phloroglucinol, Potassium Ascorbyl Tocopheryl Phosphate, Thiodiglycolamide, Potassium Sulfite, Propyl Gallate, Rosmarinic Acid, Rutin, Sodium Ascorbate, Sodium Ascorbyl/Cholesteryl Phosphate, Sodium Bisulfite, Sodium Erythorbate, Sodium Metabisulfide, Sodium Sulfite, Sodium Thioglycolate, Sorbityl Furfural, Tea Tree (Melaleuca Aftemifolia) Oil, Tocopheryl Acetate, Tetrahexyldecyl Ascorbate, Tetrahydrodiferuloylmethane, Tocopheryl Linoleate/Oleate, Thiodiglycol, Tocopheryl Succinate, Thiodiglycolic Acid, Thioglycolic Acid, Thiolactic Acid, Thiosalicylic Acid, Thiotaurine, Retinol, Tocophereth-5, Tocophereth-10, Tocophereth-12, Tocophereth-18, Tocophereth-50, Tocopherol, Tocophersolan, Tocopheryl Linoleate, Tocopheryl Nicotinate, Tocoquinone, o-Tolyl Biguanide, Tris(Nonylphenyl)Phosphite, Ubiquinone, and Zinc Dibutyldithiocarbamate.

Some examples of antiperspirant agents and deodorant agents are Aluminum Chloride, Aluminum Zirconium Tetrachlorohydrex GLY, Dichloro-m-Xylenol, Aluminum Chlorohydrate, Aluminum Zirconium Tetrachlorohydrex PEG, Aluminum Chlorohydrex, Aluminum Zirconium Tetrachlorohydrex PG, Aluminum Chlorohydrex PEG, Aluminum Zirconium Trichlorohydrate, Domiphen Bromide, Aluminum Chlorohydrex PG, Aluminum Zirconium Trichlorohydrex GLY, Hexachlorophene, Aluminum Dichlorohydrate, Ammonium Phenolsulfonate, Ketoglutaric Acid, Aluminum Dichlorohydrex PEG, Benzalkonium Bromide, Lauryl Isoquinolinium Bromide, Aluminum Dichlorohydrex PG, Benzalkonium Cetyl Phosphate, Laurylpyridinium Chloride, Aluminum Lactate, Benzalkonium Chloride, Methylbenzethonium Chloride, Aluminum Phenolsulfonate, Benzalkonium Saccharinate, Phenol, Aluminum Sesquichlorohydrate, Benzethonium Chloride, Sodium Bicarbonate, Aluminum Sesquichlorohydrex PEG, Bromochlorophene, Sodium Phenolsulfonate, Aluminum Sesquichlorohydrex PG, Cetylpyridinium Chloride, Tricloban, Aluminum Sulfate, Chlorophyllin-Copper Complex, Triclosan, Aluminum Zirconium Octachlorohydrate, Chlorothymol, Zeolite, Aluminum Zirconium Octachlorohydrex GLY, Zinc Lactate, Aluminum Zirconium Pentachlorohydrate, Cloflucarban, Zinc Phenolsulfonate, Aluminum Zirconium Pentachlorohydrex GLY, Dequalinium Chloride, Zinc Ricinoleate, Aluminum Zirconium Tetrachlorohydrate, and Dichlorophene.

Some examples of cosmetic biocides are Aluminum Phenolsulfonate, Ammonium Phenolsulfonate, Bakuchiol, Benzalkonium Bromide, Benzalkonium Cetyl Phosphate, Benzalkonium Chloride, Benzalkonium Saccharinate, Benzethonium Chloride, Potassium Phenoxide, Benzoxiquine, Benzoxonium Chloride, Bispyrithione, Boric Acid, Bromochlorophene, Camphor Benzalkonium Methosulfate, Captan, Cetalkonium Chloride, Cetearalkonium Bromide, Cetethyldimonium Bromide, Cetrimonium Bromide, Cetrimonium Chloride, Cetrimonium Methosulfate, Cetrimonium Saccharinate, Cetrimonium Tosylate, Cetylpyridinium Chloride, Chloramine T, Chlorhexidine, Chlorhexidine Diacetate, Chlorhexidine Digluconate, Chlorhexidine Dihydrochloride, p-Chloro-m-Cresol, Chlorophene, p-Chlorophenol, Chlorothymol, Chloroxylenol, Chlorphenesin, Ciclopirox Olamine, Climbazole, Cloflucarban, Clotrimazole, Coal Tar, Colloidal Sulfur, o-Cymen-5-ol, Dequalinium Acetate, Dequalinium Chloride, Dibromopropamidine Diisethionate, Dichlorobenzyl Alcohol, Dichlorophene, Dichlorophenyl Imidazoldioxolan, Dichloro-m-Xylenol, Diiodomethyltolylsulfone, Dimethylol Ethylene Thiourea, Diphenylmethyl Piperazinylbenzimidazole, Domiphen Bromide, 7-Ethylbicyclooxazolidine, Fluorosalan, Formaldehyde, Glutaral, Hexachlorophene, Hexamidine, Hexamidine Diisethionate, Hexamidine Diparaben, Hexamidine Paraben, Hexetidine, Hydrogen Peroxide, Hydroxymethyl Dioxoazabicyclooctane, Ichthammol, Isopropyl Cresol, Lapyrium Chloride, Lauralkonium Bromide, Lauralkonium Chloride, Laurtrimonium Bromide, Laurtrimonium Chloride, Laurtrimonium Trichlorophenoxide, Lauryl Isoquinolinium Bromide, Lauryl Isoquinolinium Saccharinate, Laurylpyridinium Chloride, Mercuric Oxide, Methenamine, Methenammonium Chloride, Methylbenzethonium Chloride, Myristalkonium Chloride, Myristalkonium Saccharinate, Myrtrimonium Bromide, Nonoxynol-9 Iodine, Nonoxynol-12 Iodine, Olealkonium Chloride, Oxyquinoline, Oxyquinoline Benzoate, Oxyquinoline Sulfate, PEG-2 Coco-Benzonium Chloride, PEG-10 Coco-Benzonium Chloride, PEG-6 Undecylenate, PEG-8 Undecylenate, Phenol, o-Phenylphenol, Phenyl Salicylate, Piroctone Olamine, Sulfosuccinylundecylenate, Potassium o-Phenylphenate, Potassium Salicylate, Potassium Troclosene, Propionic Acid, PVP-Iodine, Quaternium-8, Quaternium-14, Quaternium-24, Sodium Phenolsulfonate, Sodium Phenoxide, Sodium o-Phenylphenate, Sodium Shale Oil Sulfonate, Sodium Usnate, Thiabendazole, 2,2′-Thiobis(4-Chlorophenol), Thiram, Triacetin, Triclocarban, Triclosan, Trioctyldodecyl Borate, Undecylenamidopropylamine Oxide, Undecyleneth-6, Undecylenic Acid, Zinc Acetate, Zinc Aspartate, Zinc Borate, Zinc Chloride, Zinc Citrate, Zinc Cysteinate, Zinc Dibutyldithiocarbamate, Zinc Gluconate, Zinc Glutamate, Zinc Lactate, Zinc Phenolsulfonate, Zinc Pyrithione, Zinc Sulfate, and Zinc Undecylenate.

Some examples of external analgesics are Benzyl Alcohol, Capsicum Oleoresin (Capsicum Frutescens Oleoresin), Methyl Salicylate, Camphor, Phenol, Capsaicin, Juniper Tar (Juniperus Oxycedrus Tar), Phenolate Sodium (Sodium Phenoxide), Capsicum (Capsicum Frutescens), Menthol, Resorcinol, Methyl Nicotinate, and Turpentine Oil (Turpentine).

Some examples of oral care agents are Aluminum Fluoride, Dicalcium Phosphate Dihydrate, Sodium Bicarbonate, Ammonium Fluoride, Domiphen Bromide, Sodium Chloride, Ammonium Fluorosilicate, Ferric Glycerophosphate, Sodium Fluoride, Ammonium Monofluorophosphate, Glycerin, Sodium Fluorosilicate, Ammonium Phosphate, Hexetidine, Sodium Glycerophosphate, Calcium Carbonate, Hydrated Silica, Sodium Metaphosphate, Calcium Fluoride, Hydrogenated Starch Hydrolysate, Sodium Monofluorophosphate, Calcium Glycerophosphate, Hydrogen Peroxide, Sodium Phytate, Calcium Monofluorophosphate, Hydroxyapatite, Sodium Styrene/Acrylates/Divinylbenzene, Calcium Phosphate, Magnesium Fluoride, Calcium Pyrophosphate, Magnesium Fluorosilicate, Stannous Fluoride, Cetylamine Hydrofluoride, Magnesium Glycerophosphate, Stannous Pyropbosphate, Cetylpyridinium Chloride, Manganese Glycerophosphate, Strontium Acetate, Chlorohexidine, Olaflur, Strontium Chloride, Chlorohexidine Diacetate, Phytic Acid, Tetrapotassium Pyrophosphate, Chlorohexidine Digluconate, Polyethylene, Tetrasodium Pyrophosphate, Chlorohexidine Dihydrochloride, Potassium Fluoride, Tricalcium Phosphate, Chlorothymol, Potassium Fluorosilicate, Zinc Chloride, Dequalinium Chloride, Potassium Glycerophosphate, Zinc Citrate, Diammonium Phosphate, Potassium Monofluorophosphate, Zinc Sulfate, and Dicalcium Phosphate.

Some examples of oral care drugs are Ammonium Alum, Potassium Alum, Benzyl Alcohol, Carbamide Peroxide, Elm Bark Extract, Gelatin, Glycerin, Hydrogen Peroxide, Menthol, Pectin, Phenol, Sodium Bicarbonate, Sodium Perborate, and Zinc Chloride.

Some examples of oxidizing agents are Ammonium Persulfate, Calcium Peroxide, Hydrogen Peroxide, Magnesium Peroxide, Melamine Peroxide, Potassium Bromate, Potassium Caroate, Potassium Chlorate, Potassium Persulfate, Sodium Bromate, Sodium Carbonate Peroxide, Sodium Chlorate, Sodium Iodate, Sodium Perborate, Sodium Persulfate, Strontium Dioxide, Strontium Peroxide, Urea Peroxide, and Zinc Peroxide.

Some examples of reducing agents are Ammonium Bisufite, Ammonium Sulfite, Ammonium Thioglycolate, Ammonium Thiolactate, Cystemaine HCl, Cystein, Cysteine HCl, Ethanolamine Thioglycolate, Glutathione, Glyceryl Thioglycolate, Glyceryl Thioproprionate, Hydroquinone, p-Hydroxyanisole, Isooctyl Thioglycolate, Magnesium Thioglycolate, Mercaptopropionic Acid, Potassium Metabisulfite, Potassium Sulfite, Potassium Thioglycolate, Sodium Bisulfite, Sodium Hydrosulfite, Sodium Hydroxymethane Sulfonate, Sodium Metabisulfite, Sodium Sulfite, Sodium Thioglycolate, Strontium Thioglycolate, Superoxide Dismutase, Thioglycerin, Thioglycolic Acid, Thiolactic Acid, Thiosalicylic Acid, and Zinc Formaldehyde Sulfoxylate.

An example of a skin bleaching agent is Hydroquinone.

Some examples of skin protectants are Allantoin, Aluminum Acetate, Aluminum Hydroxide, Aluminum Sulfate, Calamine, Cocoa Butter, Cod Liver Oil, Colloidal Oatmeal, Dimethicone, Glycerin, Kaolin, Lanolin, Mineral Oil, Petrolatum, Shark Liver Oil, Sodium Bicarbonate, Talc, Witch Hazel, Zinc Acetate, Zinc Carbonate, and Zinc Oxide.

Some examples of sunscreen agents are Aminobenzoic Acid, Cinoxate, Diethanolamine Methoxycinnamate, Digalloyl Trioleate, Dioxybenzone, Ethyl 4[bis(Hydroxypropyl)] Aminobenzoate, Glyceryl Aminobenzoate, Homosalate, Lawsone with Dihydroxyacetone, Menthyl Anthranilate, Octocrylene, Octyl Methoxycinnamate, Octyl Salicylate, Oxybenzone, Padimate O, Phenylbenzimidazole Sulfonic Acid, Red Petrolatum, Sulisobenzone, Titanium Dioxide, and Trolamine Salicylate.

Some examples of UV light absorbing agents are Acetaminosalol, Allatoin PABA, Benzalphthalide, Benzophenone, Benzophenone 1-12, 3-Benzylidene Camphor, Benzylidenecamphor Hydrolyzed Collagen Sulfonamide, Benzylidene Camphor Sulfonic Acid, Benzyl Salicylate, Bornelone, Bumetriozole, Butyl Methoxydibenzoylmethane, Butyl PABA, Ceria/Silica, Ceria/Silica Talc, Cinoxate, DEA-Methoxycinnamate, Dibenzoxazol Naphthalene, Di-t-Butyl Hydroxybenzylidene Camphor, Digalloyl Trioleate, Diisopropyl Methyl Cinnamate, Dimethyl PABA Ethyl Cetearyldimonium Tosylate, Dioctyl Butamido Triazone, Diphenyl Carbomethoxy Acetoxy Naphthopyran, Disodium Bisethylphenyl Tiamminotriazine Stilbenedisulfonate, Disodium Distyrylbiphenyl Triaminotriazine Stilbenedisulfonate, Disodium Distyrylbiphenyl Disulfonate, Drometrizole, Drometrizole Trisiloxane, Ethyl Dihydroxypropyl PABA, Ethyl Diisopropylcinnamate, Ethyl Methoxycinnamate, Ethyl PABA, Ethyl Urocanate, Etrocrylene Ferulic Acid, Glyceryl Octanoate Dimethoxycinnamate, Glyceryl PABA, Glycol Salicylate, Homosalate, Isoamyl p-Methoxycinnamate, Isopropylbenzyl Salicylate, Isopropyl Dibenzolylmethane, Isopropyl Methoxycinnamate, Menthyl Anthranilate, Menthyl Salicylate, 4-Methylbenzylidene, Camphor, Octocrylene, Octrizole, Octyl Dimethyl PABA, Octyl Methoxycinnamate, Octyl Salicylate, Octyl Triazone, PABA, PEG-25 PABA, Pentyl Dimethyl PABA, Phenylbenzimidazole Sulfonic Acid, Polyacrylamidomethyl Benzylidene Camphor, Potassium Methoxycinnamate, Potassium Phenylbenzimidazole Sulfonate, Red Petrolatum, Sodium Phenylbenzimidazole Sulfonate, Sodium Urocanate, TEA-Phenylbenzimidazole Sulfonate, TEA-Salicylate, Terephthalylidene Dicamphor Sulfonic Acid, Titanium Dioxide, TriPABA Panthenol, Urocanic Acid, and VA/Crotonates/Methacryloxybenzophenone-1 Copolymer.

Compositions according to the invention can be formed by combining such components in the following ranges expressed as weight percent, i.e., (i) 0.1-99.9 percent of the substituted hydrocarbyl functional siloxane fluid or the substituted hydrocarbyl functional siloxane resin; (ii) 0.1 to 99.9 percent of the cosmetic ingredient, household care ingredient, or health care ingredient; (iii) 0.1-40 percent of a cosmetic active, household care active, or health care active; and (iv) the balance to 100 percent being water, an organic solvent, a silicone solvent, or one or more optional ingredients, depending upon the particular type of composition being prepared, and its intended end use or application. Generally, such compositions can generally be prepared at room temperature, using simple propeller mixers, Brookfield counter-rotating mixers, or homogenizing mixers. No special equipment or processing conditions are typically required.

### EXAMPLES

### Example 1

### Antiperspirant Stick

An antiperspirant stick was made by simultaneously mixing the following amounts of the following ingredients using a propeller blade at 1200 rpm. The mixture was heated to 80° C. until fully melted, then cooled to 60° C. with stirring, and poured into AP stick containers.

| Ingredient | Weight Percent |
|---|---|
| Decamethylcyclopentasiloxane (D5) | 38 |
| Carbinol Fluid | 15 |
| Aluminum Zirconium | |
| Tetrachlorohydrex-Gly | 25 |
| (AZG-370, Summit Research Labs, | |
| Flemington, New Jersey) | |
| Hydrogenated Castor Oil | 5 |
| Talc | 1 |
| Stearyl Alcohol | 16 |

### Example 2

### Cosmetic Foundation

A pigment premix was made by mixing the following amounts of the following ingredients:

| Pigment Premix | Weight Percent |
|---|---|
| D5 | 50.0 |
| Caprylyl Silane treated Titanium Dioxide | 13.2 |
| Caprylyl Silane treated Red Iron Oxide | 11.4 |
| Caprylyl Silane treated Yellow Iron Oxide | 18.3 |
| Caprylyl Silane treated Black Iron Oxide | 7.1 |

Phase A was them made by mixing the following amounts of the following ingredients at 400 rpm using a dual blade emulsion mixing setup. D5 in the silicone polyether used in Phase A is decamethylcyclopentasiloxane.

| Phase A | Weight Percent |
|---|---|
| Pigment Premix | 28.5 |
| Silicone Polyether in D5 | 7.5 |
| Carbinol Fluid | 8.0 |

Phase B was them made by mixing the following amounts of the following ingredients with a magnetic stir bar.

| Phase B | Weight Percent |
|---|---|
| Water | 54.8 |
| Sodium Chloride | 1.0 |
| Polyoxyethylene (20) Sorbitan | 0.2 |
| Monolaurate (Tween 20) | |

Phase B was then slowly added to phase A using an addition funnel. The mixture was then mixed an additional 10 minutes at 1376 rpm.

### Example 3

### Fabric Softener

A fabric softener was made by simultaneously mixing the following amounts of the following ingredients using a magnetic stir bar until thoroughly blended.

| Ingredient | Weight Percent |
|---|---|
| Tetranyl L1/90 | 17.8 |
| Magnesium Chloride | 0.1 |
| Preservative (Formol) | 0.1 |
| Carbinol Fluid | 2.0 |
| Water | 80.0 |

Tetranyl L1/90 is Dihydrogenated Tallowoylethyl Hydroxyethylmonium Methosulfate, a surfactant fabric softener manufactured by Kao Corporation.

### Example 4

### Hair Gel

A hair gel was made by the following process. The ingredients in Phase A were mixed together at 500 RPM, using a dual blade set up. The mixing speed was increased to 800 RPM and Phase B was added incrementally to Phase A, and then stirred until uniform Phase C was added. The mixing speed was increased to 1376 RPM and continued for an additional 10 minutes.

| | Weight Percent |
|---|---|
| Phase A | |
| Polyacrylamide (and) Isoparaffin (and) Laureth-7 | 1.5 |
| (Sepigel 305, Seppic, Paris, France) | |
| Silicone Polyether in D5 | 2.0 |
| Glycerin | 40.0 |
| Phase B | |
| Deionized water | 46.5 |
| Phase C | |
| Carbinol Fluid | 5.0 |

### Example 5

### Anhydrous Roll-On Antiperspirant

An anhydrous roll-on antiperspirant was made by the following process. The ingredients in Phase A were mixed together at 800 RPM until homogeneous. Phase B was then slowly added to Phase A, and then mixed for an additional 15 minutes.

| | Weight Percent |
|---|---|
| Phase A | |
| D5 | 70.0 |
| Carbinol Fluid | 5.0 |
| Cyclomethicone (and) Quaternium 18 Hectorite | 3.0 |
| (and) SDA 40 (Bentone Gel VS-5, | |
| Rheox Inc., Highstown, New Jersey) | |
| 200 Proof Ethanol | 2.0 |
| Phase B | |
| Aluminum Zirconium Tetrachlorohydrex-Gly | 20.0 |

### Example 6

### Hair Conditioner

A hair conditioner was made by the following process. The water in Phase A was heated to about 5° C. and then the hydroxyethyl cellulose and cetrimonium chloride was added.

The mixture was mixed until uniform. The ingredients of phase B were mixed together and heated to 60-70° C. using a water bath. Phase C was heated to 80° C. and added to phase B while stirring at 800 RPM. The mixture was stirred for an additional 10 minutes then added to Phase A with gentle mixing and continued until uniform. The mixture was then cooled to room temperature with gentle mixing, then Phase D was added and mixed for an additional 10 minutes.

| | Weight Percent |
|---|---|
| Phase A | |
| Deionized Water | 50.0 |
| Hydroxyethyl Cellulose | 1.5 |
| (Natrosol 250 HHR, Hercules Inc., | |
| Wilmington, Delaware) | |
| Cetrimonium Chloride | 0.3 |
| (Arquad 16-29, Akzo Nobel Chemicals, Inc., | |
| Chicago, Illinois) | |
| Phase B | |
| Cetearyl Alcohol | 1.0 |
| Glyceryl stearate (and) PEG 100 stearate | 1.0 |
| (Arlacel 165, Uniqema (ICI Surfactants), | |
| Wilmington, Delaware) | |
| Carbinol Fluid | 2.0 |
| Phase C | |
| Deionized Water | 44.2 |
| Phase D | |
| Preservative, DMDM Hydantoin | 0.2 |
| (Glydant, Lonza Inc., Fair Lawn, New Jersey) | |

### Example 7

### Water-In-Oil Moisturizer

A water-in-oil moisturizer was made using the following procedure. Phase A was mixed at 300 RPM and heated to 40° C. to solubilize the petrolatum and lanolin. Phase B was made by dissolving the sodium chloride in the water and then adding glycerin and mixing until uniform. Using an addition funnel, Phase B was slowly added to Phase A while mixing at 1376 RPM. After the addition was complete, preservative was added and mixing continued for 10 minutes. The resultant cream was passed through a Gifford-Wood homogenizer for 3 minutes.

| | Weight Percent |
|---|---|
| Phase A | |
| Crosslinked Silicone Polyether | 2.0 |
| (Laurylmethicone Copolyol) | |
| Mineral Oil | 10.0 |
| Petrolatum | 2.0 |
| Lanolin | 1.0 |
| Isopropyl Myristate, (Emerest 2314, | 2.0 |
| Henkel Corp./Emery Group, | |
| Ambler, Pennsylvania) | |
| Carbinol Fluid | 1.0 |
| D5 | 5.0 |
| Phase B | |
| Glycerin | 5.0 |
| Sodium Chloride | 2.0 |
| Deionized Water | 69.8 |
| Preservative, Diazolidinyl urea & parabens, | 0.2 |
| Germaben II, International Specialty Product/ISP, | |
| Wayne, New Jersey) | |

Compositions prepared according to the invention can be used in various over-the-counter (OTC) personal care compositions, health care compositions, and household care compositions, but especially in the personal care arena. Thus, they can be used in antiperspirants, deodorants, skin creams, skin care lotions, moisturizers, facial treatments such as acne or wrinkle removers, personal and facial cleansers, bath oils, perfumes, colognes, sachets, sunscreens, pre-shave and after-shave lotions, liquid soaps, shaving soaps, shaving lathers, hair shampoos, hair conditioners, hair sprays, mousses, permanents, depilatories, hair cuticle coats, make-ups, color cosmetics, foundations, blushes, lipsticks, lip balms, eyeliners, mascaras, oil removers, color cosmetic removers, nail polishes, and powders.

Other variations may be made in compounds, compositions, and methods described herein without departing from the essential features of the invention. The embodiments of the invention specifically illustrated herein are exemplary only and not intended as limitations on their scope except as defined in the appended claims.

1. A composition comprising a guar gum free and hydroxystearic acid derivative free mixture of (i) at least one substituted hydrocarbyl functional siloxane fluid containing only a single oxyalkylene unit in any chain; and (ii) at least one cosmetic ingredient, household care ingredient, or health care ingredient, other than an active cosmetic, household care, or health care ingredient.

2. A composition according to claim 1 further comprising (iii) at least one cosmetic, household care, or health care active ingredient selected from the group consisting of antiacne agents, anticaries agents, antidandruff agents, antifungal agents, antimicrobial agents, antioxidants, antiperspirant agents, cosmetic biocides, deodorant agents, external analgesics, oral care agents, oral care drugs, oxidizing agents, reducing agents, skin bleaching agents, skin protectants, sunscreen agents, UV light absorbing agents, pigments, moisturizers, vitamins, enzymes, optical brighteners, fabric softening agents, and surfactants.

3. A composition according to claim 1 in which the substituted hydrocarbyl functional siloxane fluid has a formula selected from the group consisting of:
R₃SiO(RR¹ SiO)_{y}SiR₃,
R₃ SiO(R₂SiO)ₓ(RR¹SiO)_{y}SiR₃,
R¹R₂SiO(R₂SiO)ₓSiR₂R¹,
R¹R₂SiO(RR¹SiO)_{z}SiR₂R¹,
R¹R₂SiO(R₂SiO)ₓ(RR¹SiO)_{z}SiR₂R¹,
R¹R₂SiO(R₂SiO)ₓSiR₃,
R¹R₂SiO(RR¹SiO)_{z}SiR₃,
R¹R₂SiO(R₂SiO)ₓ(RR¹SiO)_{z}SiR₃, and cyclic siloxanes of the formula
(Me₂SiO)ₘ(MeR¹SiO)ₙ—
where R is an alkyl, cycloalkyl, alkenyl, aralkyl, or an aryl group containing 1-20 carbon atoms; R¹ is a group having one of the formulas (i)-(iv):
—(CH₂)ₐ(OCH₂CH₂)OR² (i)
—(CH₂)ₐ(OCH₂CH₂CH₂)OR² (ii)
—(CH₂)ₐ[OCH₂CH(CH₂CH₃)]OR² (iii)
—(R)_{b}OR² (iv)
in which a is 3-11; b is 1-50; R² is selected from the group consisting of hydrogen, an alkyl group, an aryl group, an arylalkyl group and an acyl group; x is 1-500, y is 1-40, z is 1-40, m is 1-6, n is 1-6, and the sum of m+n is 3-12.

4. A method of providing a cosmetic property, a household care property, or a health care property to a substrate comprising applying to the substrate a composition according to claim 1.

5. A product containing the composition of claim 1 selected from the group consisting of hairsprays, shampoos, mousses, styling gels, styling lotions, cream rinses, conditioners, hair tonics, hair dyes, hair colorants, permanent waves, bleaches, hair cuticle coats, skin cleansers, moisturizers, lipsticks, eye makeup, fingernail polish, suntan products, antiperspirants, deodorants, depilatories, household waxes, polishes, heavy duty liquid cleaners, light duty liquid cleaners, fabric softeners, laundry detergents, ironing aids, and window cleaners.

6. A composition comprising a guar gum free and hydroxystearic acid derivative free mixture of
(i) a substituted hydrocarbyl functional siloxane resin having the formula
(SiO₂)_{c}(R³SiO_{3/2})_{d}(R³₂SiO)ₑ(R³₃SiO_{1/2})_{f}{O_{1/2}SiR³₂R⁴}_{g}
where R³ is an alkyl group with 1-20 carbon atoms, a cycloalkyl group with 3-20 carbon atoms, an alkenyl group with 2-20 carbon atoms, an aralkyl group, or an aryl group;
R⁴ is a group having one of the formulas (i)-(iv):
—(CH₂)ₐ(OCH₂CH₂)OR² (i)
—(CH₂)ₐ(OCH₂CH₂CH₂)OR² (ii)
—(CH₂)ₐ[OCH₂CH(CH₂CH₃)]OR² (iii)
—(R)_{b}OR² (iv)
in which a is 3-11; b is 1-50; R² is selected from the group consisting of hydrogen, an alkyl group, an aryl group, an arylalkyl group and an acyl group; x is 1-500, y is 1-40, z is 1-40, m is 1-6, n is 1-6, and the sum of m+n is 3-12; g is 1-15,000; c, d, e, and f are mole percents such that c is less than 100, c+d is more than 0, and c+d+e+f is 100; and
(ii) at least one cosmetic ingredient, household care ingredient, or health care ingredient, other than an active cosmetic, household care, or health care ingredient.

7. A composition according to claim 6 further comprising (iii) at least one cosmetic, household care, or health care active ingredient selected from the group consisting of antiacne agents, anticaries agents, antidandruff agents, antifungal agents, antimicrobial agents, antioxidants, antiperspirant agents, cosmetic biocides, deodorant agents, external analgesics, oral care agents, oral care drugs, oxidizing agents, reducing agents, skin bleaching agents, skin protectants, sunscreen agents, UV light absorbing agents, pigments, moisturizers, vitamins. enzymes, optical brighteners, fabric softening agents, and surfactants.

8. A method of providing a cosmetic property, a household care property, or a health care property to a substrate comprising applying to the substrate a composition according to claim 6.

9. A product containing the composition of claim 6 selected from the group consisting of hairsprays, shampoos, mousses, styling gels, styling lotions, cream rinses, conditioners, hair tonics, hair dyes, hair colorants, permanent waves, bleaches, hair cuticle coats, skin cleansers, moisturizers, lipsticks, eye makeup, fingernail polish, suntan products, antiperspirants, deodorants, depilatories, household waxes, polishes, heavy duty liquid cleaners, light duty liquid cleaners, fabric softeners, laundry detergents, ironing aids, and window cleaners.

10. A composition comprising a guar gum free and hydroxystearic acid derivative free mixture of (i) at least one substituted hydrocarbyl functional siloxane fluid; and (ii) at least one cosmetic ingredient, household care ingredient, or health care ingredient, other than an active cosmetic, household care, or health care ingredient.

11. A composition according to claim 10 wherein the substituted hydrocarbyl functional siloxane fluid is a monoallylether modified siloxane.

12. A composition according to claim 11 further comprising (iii) at least one cosmetic, household care, or health care active ingredient selected from the group consisting of antiacne agents, anticaries agents, antidandruff agents, antifungal agents, antimicrobial agents, antioxidants, antiperspirant agents, cosmetic biocides, deodorant agents, external analgesics, oral care agents, oral care drugs, oxidizing agents, reducing agents, skin bleaching agents, skin protectants, sunscreen agents, UV light absorbing agents, pigments, moisturizers, vitamins, enzymes, optical brighteners, fabric softening agents, and surfactants.
